(19) United States
(12) Patent Application Publication
(10) Pub. No.: US 2023/0313232 A1
(43) Pub. Date: Oct. 5, 2023

(54) METHODS AND SYSTEMS FOR REAL-TIME, CONTINUOUS PRODUCTION OF NON-VIRAL CARRIER NUCLEIC ACID PARTICLES

(71) Applicant: CORNING INCORPORATED, Corning, NY (US)

(72) Inventors: Carine Cerato-Noyerie, Samoreau (FR); Samantha Sophie Catherine Haller, Paris (FR); Davis Henry, Fontaine le Port (FR); Marylène Denise Madeleine Pécheul, Chaintreaux (FR); Corinne Walerack, Veneux les sablons (FR)

(21) Appl. No.: 18/175,790

(22) Filed: Feb. 28, 2023

Related U.S. Application Data
(60) Provisional application No. 63/314,726, filed on Feb. 28, 2022.

(51) Int. Cl. C12N 15/87; C12M 1/00

(52) U.S. Cl. CPC C12N 15/87; C12M 29/10

(57) ABSTRACT

Methods and systems are provided for transfecting cells using real-time, continuous transfection of cells. In some aspects, the methods can be applied for the continuous production of non-viral vector nucleic acid complexes. The systems and methods include a passive mixing fluidic module with at least two inlets, a plurality of mixing elements, and an outlet to provide a continuous flow of transfection complexes to a cell reactor. The transfection agent and nucleic acid are passively mixed and then provided to cells in a continuous flow of cell medium. In some aspects, the flow of cell medium perfusing through the cell reactor recirculates. The system and the methods of the present disclosure provide for highly reproducible and scalable transfection with a low coefficient of variation.

### CROSS-REFERENCE TO RELATED APPLICATIONS

This application claims priority to U.S. Provisional Pat. Application 63/314,726, filed Feb. 28, 2022, the content of which is hereby incorporated by reference in its entirety.

### TECHNICAL FIELD

This disclosure generally relates to production of non-viral carrier nucleic acid particles. In particular, the present disclosure relates to methods and systems for continuous, scalable production of non-viral carrier nucleic acid particles to transfect cells in a culture vessel or bioreactor.

### BACKGROUND

Non-viral vector nucleic acid complexes for cell transfection, such as polyplexes, are typically prepared by bulk mixing and usually require an incubation time of 5 to 30 minutes to form stable DNA-transfection agent complexes. Such an incubation time has been considered as a standard for conventional technologies. However, the incubation time of bulk mixing does not allow for production of DNA-transfection complexes in a continuous manner, thereby resulting in a discontinuous transfection process. Non-continuous transfection processes may be less reliable, less reproducible from batch-to-batch, and may also present scale-up challenges.

Examples of bulk mixing are reported in “Guide for DNA Transfection in iCELLis® 500 and iCELLis 500+ Bioreactors for Large Scale Gene Therapy Vector Manufacturing,” where two DNA transfection methods are provided. In such methods, the plasmids and the polyethyleneimine (PEI or PEIpro) are placed into two separate single-use bags. In one method, bulk mixing is performed using gravity to add the PEIpro to the DNA. In a second method, bulk mixing is performed using a peristaltic pump to add the PEIpro to the DNA. The iCELLis® guide further recommends gently mixing the bag manually, which may present difficulty when handling large volumes. Thus, the bulk mixing process is non-continuous and prone to variability. Furthermore, Legmann et al. (Transient Transfection at Large Scale for Clinical AAV9 Vector Manufacturing, B Poster Content as Presented at ISCT 2020 Virtual, May 2020) reports that the incubation time for complex formation by bulk mixing via the iCELLis® bioreactor is 25 minutes, thereby rendering the process discontinuous.

CN 104974933B reports a continuous mode transfection example, namely a device and method for long-term expression of recombinant protein in a continuous suspension bioreactor through multiple transient transfections. Although transfection is performed in a flow mode, the preparation of the plasmid/vector complex is not described but it is indicated that it requires an incubation time of at least 5 to 30 minutes to form stable DNA/transfection agent complexes. Thus, the incubation time renders the process discontinuous.

WO 2018/208960A1 reports scalable methods of creating DNA and transfection agent master mixes for transfecting cells. Methods include preparing a transfection master mix by introducing a DNA solution and a transfection agent solution into a mixing container, and incubating the transfection master mix for an incubation period during which the transfection master mix is substantially still, the incubation period being between 5 to 180 minutes. However, the incubation time renders the process non-continuous.

Lu et al. (POLYPLEX SYNTHESIS BY “MICROFLUIDIC DRIFTING” BASED THREE-DIMENSIONAL HYDRODYNAMIC FOCUSING METHOD; ACS Nano. 2014 Jan 28; 8(1): 332-339) reports preparing polyplexes in a continuous manner in microfluidic devices, namely synthesized DNA and/or polymer nanocomplexes using a 3D hydrodynamic focusing method. Lu et al. reports that the nanocomplexes prepared by the 3D focusing method have smaller size, slower aggregation rate, higher transfection efficiency, and induce similar cytotoxicity compared to the nanocomplexes prepared by bulk mixing methods. However, the throughput of such a microfluidic device reported by Lu et al. is low and requires significant numbering up to meet a volume of transfection solution required for manufacturing.

Consequently, there is a need for real-time continuous, scalable production of non-viral carrier nucleic acid particles to transfect cells cultured in a culture vessel or bioreactor.

### SUMMARY

A 1^{st} aspect of the present disclosure, either alone or in combination with any other aspect herein, concerns a method for preparing transfected eukaryotic cells comprising: operably connecting a nucleic acid solution (NAS) and a transfection agent solution (TAS) to a passive mixing module through two separate inlets, with a single outlet operably connected to a cell reactor, wherein the NAS comprises a nucleic acid at a first concentration and wherein the TAS comprises a transfection agent at a second concentration; providing the NAS at a first flow rate; providing the TAS at a second flow rate; providing a combined stream of the NAS and TAS from the single outlet to the cell reactor through a length of tubing; and, perfusing the cell reactor with cell medium from a media stock reservoir into an inlet of the cell reactor and out of an outlet of the cell reactor, wherein cells reside within the cell reactor between the inlet and the outlet.

A 2^{nd} aspect of the present disclosure, either alone or in combination with any other aspect herein, concerns the method of the 1^{st} aspect, wherein the outlet of the cell reactor provides the cell medium back to the media stock reservoir to recirculate the cell medium.

A 3^{rd} aspect of the present disclosure, either alone or in combination with any other aspect herein, concerns the method of the 1^{st} or 2^{nd} aspect, wherein the combined stream of the NAS and TAS combines with the cell medium prior to entering the inlet of the cell reactor.

A 4^{th} aspect of the present disclosure, either alone or in combination with any other aspect herein, concerns the method of the 3^{rd} aspect, wherein the combined stream of the NAS and TAS combines with the cell medium in the media stock reservoir.

A 5^{th} aspect of the present disclosure, either alone or in combination with any other aspect herein, concerns the method of the 3^{rd} aspect, wherein the combined stream of the NAS and TAS combines with the cell medium at a port prior to the inlet of the cell reactor, wherein the port is configured to receive the combined stream of the NAS and TAS and a stream of cell medium from the media stock reservoir.

A 6^{th} aspect of the present disclosure, either alone or in combination with any other aspect herein, concerns the method of the 1^{st} aspect, wherein the outlet of the cell reactor is operably linked to the media stock reservoir to returning the cell medium thereto.

A 7^{th} aspect of the present disclosure, either alone or in combination with any other aspect herein, concerns the method of the 1^{st} aspect, wherein the length of tubing is configured to provide a residency time of between 1 and 30 minutes before the combined stream reaches the cell reactor.

An 8^{th} aspect of the present disclosure, either alone or in combination with any other aspect herein, concerns the method of the 1^{st} aspect, wherein the passive mixing fluidic module comprises a Y or a T junction.

A 9^{th} aspect of the present disclosure, either alone or in combination with any other aspect herein, concerns the method of the 1^{st} aspect, wherein the passive mixing fluidic module comprises a heart shaped mixing chamber, wherein the two separate inlets reside at top of the heart and the outlet resides at the bottom point of the heart.

A 10^{th} aspect of the present disclosure, either alone or in combination with any other aspect herein, concerns the method of the 9^{th} aspect, wherein the heart-shaped mixing chamber further comprises a U-bend obstruction therein.

An 11^{th} aspect of the present disclosure, either alone or in combination with any other aspect herein, concerns the method of the 9^{th} or 10^{th} aspect, wherein the passive mixing fluidic module comprises at least one additional heart shaped mixing chamber configured to receive the outlet of the preceding heart shaped mixing chamber.

A 12^{th} aspect of the present disclosure, either alone or in combination with any other aspect herein, concerns the method of the 11^{th} aspect, wherein the passive mixing fluidic module comprises a plurality of heart shaped mixing chambers in a series.

A 13^{th} aspect of the present disclosure, either alone or in combination with any other aspect herein, concerns the method of the 1^{st} aspect, wherein the first flow rate and the second flow rate are the same.

A 14^{th} aspect of the present disclosure, either alone or in combination with any other aspect herein, concerns the method of the 1^{st} or 13^{th} aspect, wherein the first concentration and second concentration are configured to provide a ratio of mass of nucleic acid to weight of transfection agent of between 4:1 to 1:4.

A 15^{th} aspect of the present disclosure, either alone or in combination with any other aspect herein, concerns the method of the 14^{th} aspect, wherein the ratio is 1:2.

A 16^{th} aspect of the present disclosure, either alone or in combination with any other aspect herein, concerns the method of the 1^{st}, 14^{th}, or 15^{th} aspect, wherein the nucleic acid comprises a plasmid.

A 17^{th} aspect of the present disclosure, either alone or in combination with any other aspect herein, concerns the method of the 16^{th} aspect, wherein the plasmid encodes an adeno-associated virus (AAV).

An 18^{th} aspect of the present disclosure, either alone or in combination with any other aspect herein, concerns the method of the 17^{th} aspect, wherein the AAV is genetically modified to express a peptide of interest.

A 19^{th} aspect of the present disclosure, either alone or in combination with any other aspect herein, concerns the method of the 17^{th} aspect, wherein the AAV is genetically modified to express a nucleic acid of interest.

A 20^{th} aspect of the present disclosure, either alone or in combination with any other aspect herein, concerns the method of the 1^{st}, 13^{th}, 14^{th}, or 17^{th} aspect, wherein the transfection agent solution comprises polyethylenimine (PEI), poly(propylene imine), DEAE-Dextran, polyarginine, dendrimers, calcium phosphate, ionizable or cationic lipids, lipid-like lipidoids, or combinations thereof.

A 21^{st} aspect of the present disclosure, either alone or in combination with any other aspect herein, concerns the method of the 20^{th} aspect, wherein the transfection agent comprises PEI.

A 22^{nd} aspect of the present disclosure, either alone or in combination with any other aspect herein, concerns the method of the 1^{st} aspect, wherein the combined stream is continuously perfused through the cell reactor for a period of time of between 30 minutes and 24 hours.

A 23^{rd} aspect of the present disclosure, either alone or in combination with any other aspect herein, concerns a system for providing continuous transfection of cells, comprising: a nucleic acid solution (NAS) containment and a transfection agent solution (TAS) containment; a passive mixing fluidic module comprised of two separate inlets and a single outlet, wherein a first inlet is operably connected to the NAS containment, the second inlet is operably connected to the TAS containment; a cell reactor comprised of an inlet and an outlet with one or more cells therebetween, wherein the outlet of the passive mixing fluidic module is operably connected to the inlet of the cell reactor; and, a media stock reservoir comprised of an inlet, an outlet, and a cell medium, wherein the outlet of the media stock reservoir is operably connected to the inlet of the cell reactor and the inlet of the media stock reservoir is operably connected to the outlet of the cell reactor.

A 24^{th} aspect of the present disclosure, either alone or in combination with any other aspect herein, concerns the system of the 23^{rd} aspect, wherein the outlet of the passive mixing fluidic module is operably connected to the inlet of the cell reactor through a length of tubing.

A 25^{th} aspect of the present disclosure, either alone or in combination with any other aspect herein, concerns the system of the 24^{th} aspect, wherein the length of tubing is configured to provide a residency time for a combined stream of the NAS and the TAS of from about 1 to about 30 minutes before reaching the cell reactor.

A 26^{th} aspect of the present disclosure, either alone or in combination with any other aspect herein, concerns the system of the 23^{rd} aspect, wherein the outlet of the passive mixing fluidic module is operably connected to the inlet of the cell reactor via the media stock reservoir.

A 27^{th} aspect of the present disclosure, either alone or in combination with any other aspect herein, concerns the system of the 26^{th} aspect, wherein the outlet of the passive mixing fluidic module operably connects to a port at the outlet of the cell reactor to allow a TAS and NAS combined solution to join the flow of cell medium to the inlet of the media stock reservoir.

A 28^{th} aspect of the present disclosure, either alone or in combination with any other aspect herein, concerns the system of the 26^{th} aspect, wherein the length of tubing operably connects to a second inlet of the media stock reservoir to allow a TAS and NAS combined solution to mix with the cell medium prior to reaching the inlet of the cell reactor.

A 29^{th} aspect of the present disclosure, either alone or in combination with any other aspect herein, concerns the system of the 23^{rd} aspect, wherein the length of tubing operably connects to a port at the inlet of the cell reactor to allow a TAS and NAS combined solution to join the flow of cell medium from the outlet of the media stock reservoir.

A 30^{th} aspect of the present disclosure, either alone or in combination with any other aspect herein, concerns the system of the and of the 23^{rd} to 29^{th} aspects, further comprising a first pump configured to pump NAS solution from the NAS containment toward the outlet of the passive mixing fluidic module and a second pump configured to pump TAS solution from the TAS containment toward the outlet of the passive mixing fluidic module.

A 31^{st} aspect of the present disclosure, either alone or in combination with any other aspect herein, concerns the system of the 30^{th} aspect, further comprising a third pump configured to flow the cell medium from the outlet of the media stock reservoir toward the inlet of the cell reactor along the length of tubing.

A 32^{nd} aspect of the present disclosure, either alone or in combination with any other aspect herein, concerns the system of the 30^{th} aspect, further comprising a third pump configured to flow the cell medium from the outlet of the cell reactor toward the inlet of the media stock reservoir.

A 33^{rd} aspect of the present disclosure, either alone or in combination with any other aspect herein, concerns the system of the 23^{rd} aspect, wherein the passive mixing fluidic module comprises a microfluidic mixing device.

A 34^{th} aspect of the present disclosure, either alone or in combination with any other aspect herein, concerns the system of the 33^{rd} aspect, wherein the microfluidic mixing device comprises a plurality of mixing elements.

A 35^{th} aspect of the present disclosure, either alone or in combination with any other aspect herein, concerns the system of the 34^{th} aspect, wherein the plurality of mixing elements comprises heart-shaped mixing elements connected in a series.

A 36^{th} aspect of the present disclosure, either alone or in combination with any other aspect herein, concerns the system of the 33^{rd} aspect, wherein the microfluidic mixing device comprises a continuous microfluidic flow reactor.

A 37^{th} aspect of the present disclosure, either alone or in combination with any other aspect herein, concerns the system of the 33^{rd} aspect, wherein the microfluidic mixing device is formed of a polymer material.

A 38^{th} aspect of the present disclosure, either alone or in combination with any other aspect herein, concerns the system of the 33^{rd} aspect, wherein the microfluidic mixing device is formed of a glass material.

A 39^{th} aspect of the present disclosure, either alone or in combination with any other aspect herein, concerns the system of the 23^{rd} aspect, wherein the passive mixing fluidic module comprises a plurality of microfluidic mixing devices arranged in series.

A 40^{th} aspect of the present disclosure, either alone or in combination with any other aspect herein, concerns the system of the 39^{th} aspect, wherein each microfluidic mixing device comprises a heart-shaped geometry.

A 41^{st} aspect of the present disclosure, either alone or in combination with any other aspect herein, concerns the system of the 40^{th} aspect, wherein each microfluidic mixing device further comprises a U-bend obstruction.

A 42^{nd} aspect of the present disclosure, either alone or in combination with any other aspect herein, concerns the system of the 39^{th} aspect, wherein the microfluidic mixing device comprises an in-line static mixer element.

A 43^{rd} aspect of the present disclosure, either alone or in combination with any other aspect herein, concerns the system of the 42^{nd} aspect, wherein the first inlet, the second inlet and the outlet of the passive mixing fluidic module meet at a T or Y junction.

A 44^{th} aspect of the present disclosure, either alone or in combination with any other aspect herein, concerns the system of the 42^{nd} aspect, wherein the in-line static mixer element is connected after the T-junction or Y-junction.

A 45^{th} aspect of the present disclosure, either alone or in combination with any other aspect herein, concerns the system of the 44^{th} aspect, wherein tubing connected upstream the T-junction or Y-junction comprises double-Y tubing.

A 46^{th} aspect of the present disclosure, either alone or in combination with any other aspect herein, concerns the system of the 23^{rd} aspect, further comprising aseptic connectors.

A 47^{th} aspect of the present disclosure, either alone or in combination with any other aspect herein, concerns the system of the 23^{rd} aspect, wherein the TAS comprises a transfection agent chosen from polyethylenimine (PEI), poly(propylene imine), DEAE-Dextran, polyarginine, dendrimers, calcium phosphate, ionizable or cationic lipids, lipid-like lipidoids, or combinations thereof.

A 48^{th} aspect of the present disclosure, either alone or in combination with any other aspect herein, concerns the system of the 23^{rd} aspect, wherein the NAS comprises a nucleic acid comprised of a non-viral vector.

A 49^{th} aspect of the present disclosure, either alone or in combination with any other aspect herein, concerns the system of the 23^{rd} aspect, wherein the NAS comprises a plasmid or a combination of plasmids encoding an adeno-associated virus (AAV).

A 50^{th} aspect of the present disclosure, either alone or in combination with any other aspect herein, concerns the system of the 49^{th} aspect, wherein the AAV is genetically modified to express a peptide of interest.

A 51^{st} aspect of the present disclosure, either alone or in combination with any other aspect herein, concerns the system of the 49^{th} aspect, wherein the AAV is genetically modified to express a nucleic acid of interest.

A 52^{nd} aspect of the present disclosure, either alone or in combination with any other aspect herein, concerns a method for preparing a nucleic acid-transfection agent complex suspension comprising: providing a passive mixing fluidic module comprising a first and a second inlet and an outlet; providing a transfection agent solution (TAS) to the first inlet and a nucleic acid solution (NAS) to the second inlet; mixing the TAS and NAS by flowing the solutions through the passive mixing fluidic module to create a resulting suspension; and providing the resulting suspension from the outlet to a cell reactor.

A 53^{rd} aspect of the present disclosure, either alone or in combination with any other aspect herein, concerns a system for preparing a nucleic acid-transfection agent complex suspension for use in transfection comprising: a passive mixing fluidic module comprising a first and second inlet, an outlet, and at least one microfluidic mixing device having a plurality of mixing elements; wherein the first inlet being operably connected to a transfection agent solution (TAS) containment and further wherein the second inlet being operably connected to a nucleic acid solution (NAS) containment; and a cell reactor comprised of an inlet and an outlet with one or more cells therebetween, wherein the outlet of the passive mixing fluidic module is operably connected to the inlet of the cell reactor.

### BRIEF DESCRIPTION OF THE DRAWINGS

FIG. 1 is a schematic of a system according to an embodiment of the disclosure.

FIG. 2A is a schematic of a system according to an embodiment of the disclosure.

FIG. 2B is a further schematic of a system according to an embodiment of the disclosure.

FIG. 3 is a mixing fluidic module according to an embodiment of the disclosure.

FIG. 4 is a passive mixing module device according to an embodiment of the disclosure.

FIG. 5 is a graph showing the hydrodynamic diameter of polyplex particles determined by DLS as a function of time once diluted in culture media according to an embodiment of the disclosure.

FIG. 6 is a graph showing polyplex size measurement and size evolution assessment during time for batch and low flow methods according to embodiments of the disclosure.

FIG. 7 is a chart showing potential zeta measurement of complexes formed with batch and low flow methods according to embodiments of the disclosure.

FIG. 8A shows images of cells adherent on mesh and expressing GFP for batch methods according to embodiments of the disclosure.

FIG. 8B shows images of cells adherent on mesh and expressing GFP for low flow methods according to embodiments of the disclosure. Levels are similar to the of the batch methods.

FIG. 9 is a chart showing flow cytometry analysis for both batch and AFR low flow methods according to embodiments of the disclosure.

FIG. 10A shows a chart depicting cAAV2 quantification for both batch and AFR low flow methods according to embodiments of the disclosure.

FIG. 10B shows a chart depicting gAAV2 quantification for both batch and AFR low flow methods according to embodiments of the disclosure.

FIG. 10C shows a chart depicting percentage of rAAV2 virions quantification for both batch and AFR low flow methods according to embodiments of the disclosure.

FIG. 11 is a chart showing polyplex size measurement and size evolution of complexes formed with batch, low flow, and T-junction methods according to embodiments of the disclosure.

FIG. 12 is a chart showing potential zeta measurement of complexes formed with batch, low flow, and T-junction methods according to embodiments of the disclosure.

FIG. 13A shows charts depicting cAAV2 quantification for both AFR low flow and T-junction methods according to embodiments of the disclosure.

FIG. 13B shows charts depicting cAAV2 quantification for both AFR low flow and T-junction methods according to embodiments of the disclosure.

FIG. 14A shows charts analysing cAAV2 production reproducibility between the AFR method and the batch method.

FIG. 14B shows charts analysing gAAV2 production reproducibility between the AFR method and the batch method.

FIG. 14C shows charts analysing the coefficient of variation between the AFR method and the batch method.

FIG. 15A shows charts representing cAAV2 quantification according to the site of polyplex injection into the FBR.

FIG. 15B shows charts representing gAAV2 quantification according to the site of polyplex injection into the FBR.

FIG. 16A shows charts presenting cAAV2 quantification in regards to polyplex maturation time (incubation time for the batch method and tubing length or residence time for the AFR method).

FIG. 16B shows charts presenting gAAV2 quantification in regards to polyplex maturation time (incubation time for the batch method and tubing length or residence time for the AFR method).

### DETAILED DESCRIPTION

The present disclosure relates to systems and methods to provide continuous nucleic acid-transfection agent complex suspensions to cells in vitro, such as eukaryotic cells. In some aspects, the systems and the methods described herein can be utilized to provide for preparation of nucleic acid-transfection agent complex (or “transfection complex”) suspension in a continuous and highly reproducible manner, providing consistency from batch to batch. The systems and the methods as set forth herein are further scalable, such that continuous nucleic acid-transfection agent complexes may be provided to eukaryotic cells in volumes ranging from microliters, to milliliters to liters to hundreds and thousands of liters. In aspects, the present disclosure provides systems and methods for transfecting cells through providing real-time, continuous production of transfection complexes and introducing the nucleic acid complexes to eukaryotic cells in culture. In some aspects, the present disclosure concerns systems and methods that utilize flow rate to both continuously prepare transfection complexes and provide the same to eukaryotic cells to uptake. In some aspects, the system and the methods herein provide a specific setup for delivering the non-viral carrier nucleic complexes to a culture vessel in a continuous mode. The system and methods of the present disclosure may be used to produce large-scale recombinant protein synthesis by transient transfection and are particularly suited to the production of viral particles, such as adeno-associated viruses (AAVs) and lentiviruses (LVs). Some aspects of the present disclosure are directed to providing a passive mixing module to practice the transfection complex preparation. Methods and devices of the present disclosure allow for well-controlled transfection, while also reducing batch-to-batch variability and allowing seamless scale-up.

As described herein, the systems and methods of the present disclosure provide for transfecting cells using real-time, continuous production of transfection complexes. The continuous production of non-viral carrier nucleic acid particles is obtained by intimately mixing the transfection agent(s) and the nucleic acid(s), such as plasmids, within a passive mixing fluidic module to create a non-viral carrier nucleic acid particle suspension or transfection complex.

In aspects, the system and method include a nucleic acid solution (NAS) and a transfection agent solution (TAS). In aspects, the system and the methods described herein concern the passive mixing of the NAS and the TAS and the introduction of formed transfection complexes thereof into a cell reactor with cells therein to be transfected. In aspects, the nucleic acids are provided in the respective NAS at a desired concentration. In other aspects, the transfection agent is provided in the respective TAS at a desired concentration. In some aspects, the nucleic acid concentration and the transfection agent concentration are established with respect to the concentration of the other. For example, in some non-limiting aspects, it may be desirable to have a ratio of 2:1 (by weight) of transfection agent to nucleic acid or vice versa. As such, the concentrations of the respective solutions can be adjusted to achieve such. To continue with the example of a 2:1 ratio, assuming both come into contact with each other as described herein at the same rate, the concentration of one solution should be twice as much as the other. It will also be appreciated the introducing the two solutions to bring them into contact with each other as described herein at different rates can similarly affect concentration.

In some aspects, the nucleic acid:transfection agent is to be established at a ratio by mass of from about 9:1 to about 1:9, including about 8:1, 7:1, 6:1, 5:1, 4:1, 3:1, 2:1, 1:1, 1:2, 1:3, 1:4, 1:5, 1:6, 1:7, and 1:8.

In some aspects, the nucleic acid:transfection agent is established at a ratio of 1:1 to 1:4, including 1:1.25, 1:1.5, 1:1.75, 1:2, 1:2.25, 1:2.5, 1:2.75, 1:3, 1:3.25, 1:3.5, and 1:3.75. In some aspects, the nucleic acid:transfection agent is established at a ratio of 1:0.25 to 1:2, including 1:0.5, 1:0.75, 1:1, 1:1.25, 1:1.5, and 1:1.75. In aspects, the nucleic acid may include a plasmid or combination of plasmids. In some aspects, the transfection agent may include PEI. In some aspects, the plasmid(s) may encode a virion or a protein thereof. In some aspects, the transfection agent may be a viral encoding-plasmid transfection agent, such as FectroVIR® (POLYPLUS).

In aspects, the system and the methods of the present disclosure include contacting the nucleic acid solution (NAS) and the transfection agent solution (TAS) with each other to allow for the formation of a transfection complex. In aspects, the NAS and the TAS are pumped or injected or drawn into a passive mixing fluidic module such that they are allowed to come into contact with each other. In some aspects, the NAS and the TAS are introduced into the passive mixing fluidic module at the same or a similar rate. As used herein, similar may be understood to refer to being within about 10% of the comparative value. In other aspects, the NAS and TAS are introduced at different flow rates.

In aspects, the NAS and the TAS are operably connected to separate inlets of a passive mixing fluidic module such that the two solutions flow together therein and passively mix. In some aspects, the NAS may flow into the passive mixing fluidic module through a first inlet and the TAS may flow through a second inlet. Such connections may be through tubing or the like and may further include tubing connectors. In some aspects, a first pump may control the flow of the NAS. In some aspects, a second pump may control the flow of the TAS. It will be apparent that the flow rate of the NAS (flow_{NAS}) and the flow rate of the TAS (flow_{TAS}) may be adjusted as well as the concentrations of nucleic acid and transfection agent to control the passive mixing together thereof.

In aspects, the NAS and TAS flow through a passive mixing fluidic module of a single channel or line, such as through a “Y” or “T” junction with two input lines and a single output line. The output line with both NAS and TAS combined then proceeds to a cell culture vessel or reactor. The flow rate from the outlet of the passive mixing fluidic module (flowₒᵤₜₗₑₜ) is the sum of flow_{NAS} and flow_{TAS}.

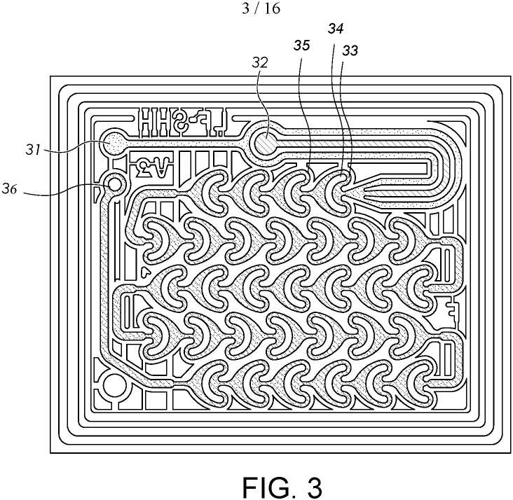

In aspects, the passive mixing fluidic module is a chamber or a series of chambers. In some aspects, the chamber features two input lines feeding into the chamber that is of a heart-like shape with an output line at the point of the heart. The volume of the chamber in relation to the diameter of the input lines creates some mild turbulence as the increase in surface area allows for TAS and NAS to passively mix therein. In some aspects, the heart-like shape includes one or more obstructions or cut-outs therein to provide additional surfaces to create turbulence and enhance passive mixing. For example, as depicted in FIG. 3, a “U-bend” obstruction is provided to increase turbulence and mixing. In aspects where at least two chambers are used in a series, the NAS and TAS flow into the chamber at the top and exit at the point into the top of the next chamber, where the output from the first chamber is divided into two streams at the top of the heart, only to combine again to a single flow at the base thereof, thereby providing additional passive mixing as the NAS/TAS repeatedly combine and divide through multiple chambers. Accordingly, in some aspects, fluidic modules may be arranged, such as in series, to increase the residence time within the system for the transfection complex to fully form.

In aspects, the passive mixing fluidic module or mixing device is a microfluidic mixing device. One non-limiting example of a microfluidic mixing device comprises the advanced flow reactor (AFR) (available from CORNING INCORPORATED, Corning, NY) that provides seamless scale-up by design.

In some aspects, more than one NAS/TAS set up can be utilized to provide transfection complexes to the cells. For example, as the volume of the system increases, the volume of NAS and TAS will also increase. However, the passive mixing thereof may remain on a smaller scale to allow for the transfection complexes to efficiently form. Accordingly, it is a further aspect of the present disclosure to include additional feeds into the cell reactor of NAS/TAS mixtures. Rather than increase the volume of the feed, providing multiple NAS and TAS chambers, each mixing together separately allows a further opportunity for the scale-up in volume. In some aspects, a first mixed NAS/TAS solution from a first fluidic module and second mixed NAS/TAS solution from a second fluidic module may enter a third fluidic module. That is to say, the output from one fluidic module may be combined with the output of a second fluidic module, and so on to increase the volume of formed complexes prior to entering the cell reactor.

In aspects, in addition to passive mixing, the nucleic acid and the transfection agent require time to form the transfection complex prior to being able to effectively transfect the cells. In some aspects, the systems and method of the present disclosure include one or more approaches to increase residency time within the system between the point of initial contact between the NAS and the TAS and when the combined solution with the transfection complexes formed therein enters the cell reactor. In some aspects, providing a length of tubing between the outlet of the passive mixing module and the cell reactor and/or media stock reservoir as described herein offers an opportunity to control the residency time of the transfection complex prior to initiating contact with the cells. In aspects, it is a function of the flow rate, tubing length, and internal tubing volume/internal tubing diameter that can control the residency time. By increasing the length of tubing, the residency time increases. As set forth in the working examples, increasing the residency time from 1 to 3 minutes allowed for improved transfection of cells in the cell reactor. In some aspects, prior to transfecting the cells, a flow rate for the TAS and NAS will be selected. As discussed herein, the tubing diameter may also affect whether the flow is laminar or turbulent. Based on these parameters, it is therefore possible to select a length of tubing sufficient to provide a desired residency time. For example, ass described in the working examples herein, a flow rate of about 10 mL/min benefitted from a length of tubing that increased residency time to about 3 minutes prior to coming into contact with the cells. For higher flow, such as in scaling the system up, longer tubing will be needed. For example, as set forth in the working examples, the G1 apparatus can operate at a flow rate of 150 mL/min, which would require the tubing length to be increased to allow for the desired residency time.

In aspects, the system and methods of the present disclosure concern a cell reactor perfused with a cell medium from a media stock, herein also referred to as a media stock reservoir. It will be appreciated that the term “reservoir” is not to be viewed as limiting with respect to the materials or geometries thereof. The cell medium is provided from the media stock reservoir through an outlet thereof to an inlet of the cell reactor. The cell medium flows across cells within the cell reactor and exits via an outlet and back to the media stock reservoir via an inlet. The cell medium in such an arrangement can continuously perfuse the cells.

In some aspects, the transfection complexes from the passive mixing fluidic module are introduced to the cell medium, which in turn allows the transfection complexes to reach cells of the cell reactor. In other aspects, the transfection complexes are provided to the cell reactor, which in turn allows the transfection complexes to perfuse and circulate with the cell medium.

In aspects, following passive mixing, the (final) outlet stream from the passive mixing fluidic module flows into a media stock container/reservoir. In some aspects, flow from the cell reactor may join the outlet stream from the cell reactor prior to reaching the media stock container or reservoir. In some aspects, flow from the cell reactor through the outlet thereof may be through a flow restricting mechanism, such as a one-way valve to prevent the nucleic acid complex from entering the cell reactor prior to mixing with cell media in the media stock reservoir/container. In aspects, the outlet stream from a passive mixing fluidic module flows into a media stock reservoir and mixes with a cell medium therein. The cell medium within the media stock reservoir is in a perfusion loop with the cell reactor. Accordingly, adding the outlet stream from the passive mixing fluidic module to the cell medium allows for transfection complexes therein to continuously perfuse through the cell reactor. In some aspects, the outlet stream from a passive mixing fluidic module directly flows into a cell reactor with cell media circulating therethrough by operable connections to a media stock reservoir/container.

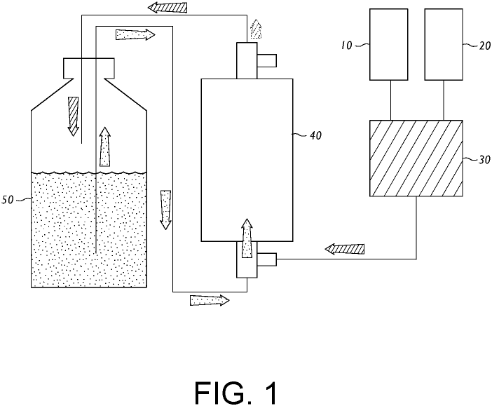

The following references to the figures are for illustrative purposes only to exemplify arrangements that allow for continuous perfusion of the cell reactor with transfection complexes. Turning to FIG. 1, depicted is an arrangement showing a nucleic acid solution (NAS) 10 and a transfection agent solution (TAS) 20 flowing separately into a passive mixing fluidic module 30 such as a T-junction, a Y junction, or an AFR module. The output stream from the passive mixing fluidic module 30 proceeds to a port on a cell reactor 40 where it is combined with a further pumped stream from a media stock reservoir 50. Although not depicted, it will be apparent that the flow from the passive mixing fluidic module 30 proceeds to the port on the cell reactor 40 along a length of tubing. The flow out of the cell reactor 40 recirculates back to the media stock reservoir 50, thereby allowing the nucleic acid-transfection agent complex to recirculate back into the cell reactor 40 from the media stock reservoir 50.

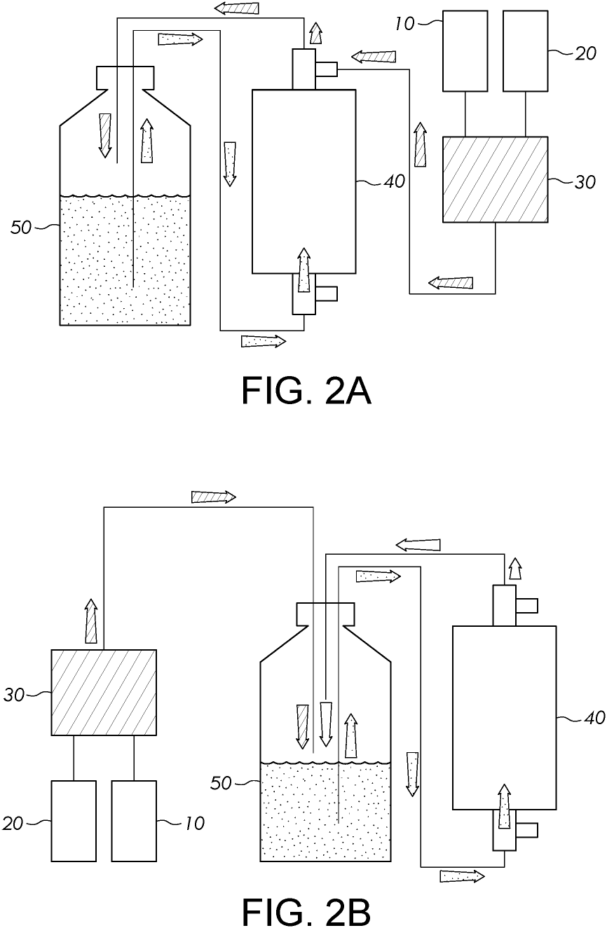

FIG. 1 shows one embodiment of the system where non-viral vector nucleic acid complex suspension is discharged at the outlet of the passive mixing fluidic module and directly injected into the inlet of the vessel containing the cells to be transfected. FIGS. 2A and 2B show two embodiments of the system where the non-viral vector/nucleic acid complex suspension may be injected directly in the media reservoir when the vessel is under perfusion.

Turning to FIG. 2A, the output stream from the passive mixing fluidic module 30 flows to a port on the top of the cell reactor 40 and is mixed therein with the cell media. Although not depicted, it will be apparent that the flow from the passive mixing fluidic module 30 proceeds to the port on the top of the cell reactor 40 along a length of tubing. The mixture can then be pumped from the media stock reservoir 50 into the cell reactor 40. The output stream from the cell reactor 40 may further join the output stream from the passive mixing fluidic module 30 at the port of the cell reactor 40 to allow the media of the media stock reservoir 50 to recirculate back to the media stock reservoir 50. The output stream of the combined nucleic acid solution (NAS) 10 and transfection agent solution (TAS) 20 accordingly also recirculates through the cell reactor 40 by virtue of being added into the media in the media stock reservoir 50.

Turning to FIG. 2B, depicted is a different point of input for the output stream from the passive mixing fluidic module. The output stream from the passive mixing fluidic module 30 flows directly into the media stock reservoir 50. Although not depicted, it will be apparent that the flow from the passive mixing fluidic module 30 proceeds to the media stock reservoir 50 along a length of tubing. The media stock reservoir 50 has a single pathway recirculating media through the cell reactor 40. Accordingly, as the transfection complex enters the media in the media stock reservoir 50, it can join the media recirculating through the cell reactor 50.

In both FIGS. 1, 2A, and 2B, the system includes a media source for recirculation from the media stock reservoir 50, a nucleic acid solution 10 (e.g., DNA solution, plasmids), a transfection agent solution 20 (e.g., PEI), a passive mixing fluidic module 30 (e.g., an AFR reactor), and a cell reactor 40 (e.g., a bioreactor, cell culture reactor, perfusion reactor).

The transfection complex suspended in the outlet stream created in the passive mixing fluidic module is continuously injected in a vessel containing the cells to be transfected and the vessel is advantageously perfused by the culture medium. It is an aspect of the system and the methods of the present disclosure that through the residency time in the tubing, the TAS and NAS are continuously flowing, yet also allowed to incubate along the length of the tubing prior to reaching the cells to be transfected. Unlike conventional methods such as batch or bulk mixing, the liquids according to methods described herein are always in motion and do not remain stationary, therefore allowing for a better homogeneity of the non-viral carrier nucleic acid particles suspension. It will also be appreciated that the cell reactor may continue to be perfused with the medium and transfection complexes therein after the NAS and/or TAS are exhausted.

Because the passive mixing module is directly connected to the cell reactor, it allows for a controlled, constant, reproducible, and continuous transfection. Moreover, because the flow setup is directly connected to the reactor using sterile connectors, it does not require large size bags, handling, or hand-mixing, such as needed for conventional batch or bulk mixing protocols.

Furthermore, methods and systems described herein may also require less transfection agent, such as PEI, than direct transfection methods, such as direct transfection methods reported in the literature which consist of injecting first the plasmids into the cell vessel and then injecting the PEI in a second step (Xie et al. ; PEI/DNA formation affects transient gene expression in suspension Chinese hamster ovary cells via a one-step transfection process; Cytotechnology. 2013 Mar; 65(2): 263-271).

Methods as described herein include intimately mixing together the TAS and the NAS, within a passive mixing fluidic module and continuously injecting the resulting suspension in the culture vessel. The transfection complex suspension is discharged at the outlet of the passive mixing fluidic module and provided either directly or via mixing with media in the media stock reservoir into the inlet of the cell reactor containing the cells to be transfected. During the injection, the culture medium may or may not be circulating through the vessel. In some aspects, the transfection complex suspension may be injected directly in the media reservoir when the vessel is under perfusion.

In aspects, the cell medium from the media stock reservoir provides for continuous perfusion of the cell reactor. In adding the NAS/TAS combined output to the cell medium, the transfection complex therein is also able to continuously circulate through the cell reactor, at least until the depletion thereof by successful transfection/cellular uptake. It is an aspect of the present disclosure that the cells in the cell reactor are continuously in contact or exposed to the transfection complexes. The systems and the methods can therefore occur over any desired period of time, such as 30 minutes to 24 hours or more. In aspects, the NAS and TAS may be exhausted, yet the circulation of the cell medium allows for transfection complexes therein continue to exert an effect. Similarly, the NAS and TAS may be replaced or refilled, thereby continuing to provide transfection complexes throughout the desired period of time. In some aspects, the cell medium may be replaced or refilled, such as by diverting the flow from the outlet of the cell reactor to a waste container and/or replacing the cell medium and/or switching the connection of the inlet of the cell reactor to a new media stock reservoir.

As set forth in examples, the system and the methods provided herein allow for reproducible transfection. While batch transfections in comparison may see some higher yield, such is not consistent or reliable. The system and the methods described herein provide a low coefficient of variation, identifying that transfection is consistent. Moreover, as identified herein, the system is scalable, allow for consistent transfection of large volumes of cells. With the data provided herein, it is also demonstrated that the system and methods of the present disclosure demonstrate about 10-fold increase in the production of recombinant virions in comparison to the standard “batch” approach. By using such viral-based systems, it is therefore possible to greatly increase production of desired peptides or nucleic acids therein.

### Passive Mixing Modules

The passive mixing fluidic module of the present disclosure includes at least two inlets and at least one outlet. In between the inlet and the outlet, the passive mixing fluidic module may include mixing element(s).

The passive mixing step is primarily achieved by flowing the TAS and NAS solutions through the passive mixing device that has no mobile elements as such mobile elements could damage the nucleic acids by mechanical shearing. In some aspects, the passive mixing fluidic module allows for the nucleic acid to remain intact. For example, supercoiled circular (SC) plasmid DNA is often subjected to fluid stress in large-scale manufacturing processes. Thus, methods and systems as described herein provide passive mixing modules, wherein no degradation of the nucleic acid by mechanical force would be expected, unlike dynamic mixers which can lead to shear-induced degradation and/or nucleic acid damage.

In some aspects, the NAS and TAS flow into a passive mixing fluidic module of a single outlet channel or line, such as through a “Y” or “T” junction with two input lines and a single output line.

The passive mixing fluidic module may comprise a microfluidic mixing device. Such mixing elements of passive mixers may have various geometries. In some aspects, the passive mixer may have heart-shaped mixing zones or heart-shaped mixing elements that feature two input lines feeding into the chamber that is of a heart-like shape with an outlet line at the point of the heart. The microfluidic mixing device may comprise a continuous microfluidic flow reactor. The continuous microfluidic flow reactor is configured for scale-up from lab bench to industrial manufacturing. In some aspects, the mixing fluidic module or microfluidic mixing device is an advanced flow reactor (AFR) fluidic module having heart-shaped mixing elements (available from Corning Incorporated, Corning, NY) (See, e.g., FIG. 3).

Turning to FIG. 3, shown is an enhanced view of the AFR arrangement of multiple passive mixing modules. The NAS enters the AFR at port 32 and the TAS enters at port 31., although the two can be readily reversed without impacting the performance of the system. The two flow paths enter a first heart shaped passive mixing module 33 where the geometry, along with the cutout obstruction in the shape of a U-bend 34 and increased surface area allow for the combined solutions to mix more thoroughly. As the flow departs the first passive mixing module 33, it directly enters a second passive mixing module 35, when the output flow is divided into two pathways and the same geometries as the first passive mixing module 33 allow for additional passive mixing. The process repeats multiple time throughout the AFR before the outlet stream then is allowed to flow either directly or indirectly into the cell reactor. It will be appreciated that the repeats of the passive mixing modules both allow for additional mixing, as well as residence time within the system before leaving the exit port 36 and entering the cell reactor, both of which can allow for the transfection complex to better assemble and increase overall transfection efficiency.

Although such types of passive mixers were originally designed for chemical synthesis, their adaption for transfection has revealed them to be particularly well suited for the preparation of transfection complexes within a continuous mode. The inlets of such fluidic modules can be readily connected, such as by using aseptic connectors, to bags or other containers containing the plasmid(s) (NAS) and the transfection agent(s) (TAS) or their precursors and the outlet of the said mixing module is easily connected to the cell reactor or culture vessel hosting the cell to be transfected.

Although a mixing fluidic module made of glass can be used, any other materials like plastics can be used provided that the surface of the material do not adsorb excessive amount of transfection agents or react with any components therein. The passive mixing fluidic module can be disposable and single use. As an example, a glass mixing module is particularly adapted to reuse, as a clean in place (CIP) protocol can be applied.

As identified above, in some aspects, the passive mixing module may comprise a passive mixer with a T-junction or Y-junction. For example, the mixing step may be performed inside appropriately shaped junctions, such “T” or “Y”, using appropriate Reynolds number and turbulent mixing.

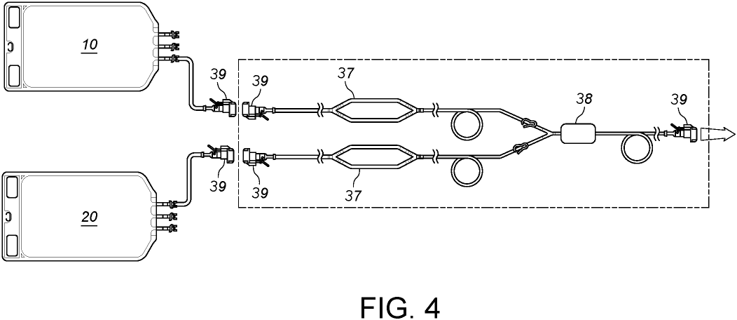

Turning to FIG. 4, depicted is a further optional arrangement for a passive mixing module that is of a “manifold-type”. Such a passive mixing module device can include at least one in-line static mixer element that is connected after the T or Y junction. The outlet of the static mixer element is connected to a tubing that can be connected to one inlet of the cell reactor (not shown). The liquids may be pushed by means of peristaltic pumps, as peristaltic pumping prevents the liquids from being in contact with the pump body and therefore preserves the sterility. The tubing connected upstream of the T or Y junction may be equipped with double-Y tubing 37 in order to reduce the pulsations which are inherent to usual peristaltic pumps. Combining the split-channel tubing with the offset rollers of two stacked peristaltic pump heads reduces pulsation by merging a pulse from one channel with a trough from the other. By doing so, the flow of NAS 10 and the flow of TAS 20 are steady when entering the passive mixing zone 38. The passive mixing module may comprise aseptic connectors, such as genderless sterile disconnectors, as shown by 39.

With regard to the system of the present disclosure, in some aspects the passive mixing module may be provided as an aseptic module packaged or stored in containers. Any suitable aseptic treatment method may be used. Any suitable container or aseptic storage may be used. As a nonlimiting example, the passive mixing module may be made aseptic by gamma irradiation treatment and stored in pouches.

In some aspects, the passive mixing module may be reusable. For example, in an embodiment, the passive mixing module may be formed of glass. When the passive mixing device, or passive mixing module, is made of glass, it can be readily cleaned by a clean-in-place (CIP) procedure and thus may be reused (CIP refers to a validated cleaning method involving automatic cleaning of interior surfaces of pipes, vessels, process equipment, and associated fittings without disassembly).

In some aspects, the passive mixing device may be reusable. For example, in an embodiment, the mixing device may be formed of glass. When the mixing device, or mixing fluidic module, is made of glass, it can be readily cleaned by the CIP procedure and thus may be reused.

In some aspects, the mixing device may be single-use or disposable. As a non-limiting example, in an aspect, the mixing device may be formed of plastic or a polymer material. A polymer or plastic-made mixing fluidic module may be ready to use with sterile connectors, would not require cleaning if used directly out of sterile packaging, and would be disposable.

### Scalability and Flow Time

The systems and methods of the present disclosure are particularly useful for production of consistent transfection complexes and consistent transfection of cells. The controlled mixing and the flow time allow for the transfection complex to be consistent in formation and uptake by cells. As seen in the working examples, transfection is achieved with minimal variance, both between transfection runs, as well as during the transfection run. For example, as set forth in the examples herein, the system and methods of the present disclosure were used to transfect HEK293 cells with an AAV-encoding plasmid(s)-PEI transfection complex. The system and methods of the present disclosure provided for a consistent 350 nm sized transfection complex with a low coefficient of variation and a higher level of virion production that was absent from a “batch” transfection control. While batch transfection methods may show some higher yields of expressed product, the system and the methods provided herein achieve superior consistency, providing for high reproducibility and confidence.

Further, the systems and methods of the present disclosure may be used to meet the demand of large-scale bioreactor needs, such as to transfect a surface area of between about 0.25 and 500 m² (or 2000 L for cell suspensions). As identified herein, the systems and methods of the present disclosure can be adapted to provide consistent transfection of cells on a small and a large scale. Such can include flow rates of 1 to 30 mL/min, 1 to 15 mL/min, 1 to 10 mL/min, 1 to 8 mL/min, 1 to 5 mL/min, 1 to 3 mL/min, 2 to 25 mL/min, 2 to 15 mL/min, 2 to 10 mL/min, 3 to 20 mL/min, 3 to 15 mL/min, 3 to 10 mL/min, 5 to 30 mL/min, 5 to 20 mL/min, 5 to 15 mL/min, 5 to 10 mL/min, 10 to 30 mL/min, 15 to 30 mL/min, 20 to 30 mL/min, 8 to 28 mL/min, 8 to 25 mL/min, 8 to 20 mL/min, and 8 to 15 mL/min. In some aspects, the system can utilize between about 10 mL and 200 mL of TAS. Such should be configured with sufficient tubing to allow for a residence time of between 1 minute and 20 minutes prior to entry in the media stock reservoir and/or the cell reactor, including about 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, and 19 minutes. In some aspects, the passive mixing module provides a surface area of between about 5 and 50 m². In such configurations, the system can utilize between about 400 mL and 4 L of TAS. Such should be configured with sufficient tubing to allow for a residence time of between 3 and 30 minutes prior to entry in the media stock reservoir and/or the cell reactor, including about 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 20, 21, 22, 23, 24, 25, 26, 27, 28, and 29 mins. In some aspects, the passive mixing module provides a surface area of between about 100 and 500 m². In such configurations, the system can utilize between about 8 L and 40 L of TAS. Such should be configured with sufficient tubing to allow for a residence time of between 4 and 20 minutes prior to entry in the media stock reservoir and/or the cell reactor, including about 4.1, 4.2, 4.3, 4.4, 4.5, 4.6, 4.7, 4.8, 4.9, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, and 19 mins.

In some aspects, the passive mixing module has an internal volume of about 0.45 mL with a flow rate of between about 2 mL/min and 10 mL/min, including 3, 4, 5, 6, 7, 8, and 9 mL/min. In some aspects, the passive mixing module has an internal volume of about 10 mL with a flow rate of between about 30 mL/min and 150 mL/min, including about 40, 50, 60, 70, 80, 90, 100, 110, 120, 130, and 140 mL/min. In some aspects, the passive mixing module has an internal volume of about 60 mL with a flow rate of between about 400 mL/min and 2000 mL/min, including about 500, 600, 700, 800, 900, 1000, 1100, 1200, 1300, 1400, 1500, 1600, 1700, 1800, and 1900 mL/min.

In some aspects, the output stream from the passive mixing fluidic module to the cell reactor and/or media stock reservoir occurs over a period of time to allow for the transfection complexes to properly form. As identified herein, the flow rate can be controlled by the combined input of the TAS and NAS, such as through a pump. Residency time can be increased by increasing the distance before the output from passive mixing fluidic module to the cell reactor/media stock reservoir. In some aspects, the flow from the output of the passive mixing fluidic module proceed along a length of tubing to the cell reactor/media stock reservoir. It will be appreciated that with the flow rate established by the NAS and TAS, the inner diameter and the length of tubing can be adjusted to reach a desired residency time. Furthermore, it will be appreciated that the inner diameter width will affect the type of flow and the Reynolds number of the flow through the tubing. For example, a wider tubing, taken into consideration with the flow rate and the viscosity of the output, might allow for a Reynolds number of below 2300, which would allow for the output to proceed as a laminar flow. Narrower tubing can increase the Reynolds number to above 4000 and allow for turbulent flow. Between the values of 2300 and 4000, the flow is neither wholly laminar nor wholly turbulent, also known as transitional flow. In some aspects, the system and methods may provide for a combination of laminar, turbulent and/or transitional flow. In some aspects, as the TAS and the NAS come into contact, transitional or turbulent flow may allow for increased mixing. In some aspects, as the transfection complex incubates during residence along the length of tubing, transitional or laminar flow may be preferred. In other aspects, turbulent flow along the length of the tubing may provide for additional mixing.

In some aspects, the systems and the methods of the present disclosure may be incorporated for scaled production and transfection with transfection complexes such as the Corning® Ascent®™ bioreactors (Corning Incorporated, Corning, NY). Table 1 shows the surface areas available to the cells to grow for different scales and the corresponding volumes of transfection mix required. Table 1 also shows the type of AFR modules (the passive mixing device) and the duration of the preparation of the complex varying from 1 minute to 20 minutes for 0.24 m² and 500 m² reactors, respectively. For further reference, the Low Flow is 72 x 62 mm with a flow rate capability of 2 to 10 mL/min, the G1 is 155 x 125 mm in size with a flow rate capability of 10 to 200 mL/min, the G3 is 310 x 250 mm with a flow rate capability of 400 to 2000 mL/min.

TABLE 1

Surface Area with respect to AFR Systems and Parameters

| Ascent FBR SA in m² | Transfection mix approximate volume (L) | AFR system | Preparation Duration (max flow rate) min |
|---|---|---|---|
| 0.24 | 0.012 | Low Flow | 1 |
| 1 | 0.08 | Low Flow | 8 |
| 2.5 | 0.2 | Low Flow | 20 |
| 5 | 0.4 | G1 | 3 |
| 20 | 1.6 | G1 | 11 |
| 50 | 4 | G1 | 27 |
| 100 | 8 | G3 | 4 |
| 200 | 16 | G3 | 8 |
| 500 | 40 | G3 | 20 |

Table 2 indicates the internal volume of the different AFR reactors and the minimum and maximum flow rates to be used for a suitable mixing in those reactors.

TABLE 2

AFR Type and Reactor Flow Rates

| AFR module | Low Flow | G1 | G3 |
|---|---|---|---|
| Internal volume mL | 0.45 | 10 | 60 |
| Flow rate min mL/min | 2 | 30 | 400 |
| Flow rate max mL/min | 10 | 150 | 2000 |

### Nucleic Acid Solution

In aspects, the NAS includes one or more nucleic acids in a solution, such as in a buffered solution, a cell media solution, water, saline, phosphate buffered saline, or similar. The nucleic acid may be of any suitable form for successful transfection, including deoxyribonucleic acid (DNA) and ribonucleic acid (RNA). The nucleic acids may in the in form of a vector, an oligonucleotide, a re-suspended lyophilized nucleic acid, an antisense sequence, a plasmid, genomic, single stranded RNA (ssRNA), double stranded RNA (dsRNA) messenger RNA, and the like. The nucleic acid may include a sequence encoding a desired gene or protein or polypeptide or fragment therein. The nucleic acid sequence may be complementary to a sequence of a desired gene or protein or polypeptide or fragment therein.

In aspects, the NAS is prepared to provide nucleic acid at a concentration of about 0.5 to 2.0 µg DNA per million cells in the cell reactor, including about 0.6, 0.7, 0.8, 0.9, 1.0, 1.1, 1.2, 1.3, 1.4, 1.5, 1.6, 1.7, 1.8, and 1.9 µg of DNA per 10⁶ cells.

In aspects, the nucleic acid is a vector, such as a viral vector or an attenuated viral vector. In some aspect, the attenuated viral vector is adeno-associated virus vector, or AAV, or lentivirus. It is understood that such may require two, three or more constructs to be transfected for the successful, controlled production of the virus that produces the protein or polypeptide desired. In aspects, the AAV is genetically modifed to produce or express a peptide or nucleic acid of interest.

In some aspects, the NAS includes a nucleic acid in a medium. As set forth herein, the medium may be a cell medium or cell culture medium. Such are described herein and generally well understood in the art. The cell medium may additionally be supplemented, such as with antibiotics, cytokines, growth factors, hormones, saccharides, or similar.

### Transfection Agents

In aspects, the TAS includes a transfection agent in a solution. Any suitable transfection agent may be used for transfecting cells in culture according to methods described herein. In aspects, the transfection agent is a polycationic agent. In some aspects, the transfection agent comprises a polymer such as polyethylenimine (PEI), poly(propylene imine), DEAE-Dextran, polyarginine, dendrimers, calcium phosphate, and ionizable or cationic lipids, lipid-like lipidoids, or combinations of transfection agents. In an embodiment, the transfection agent comprises PEI. As a nonlimiting example, methods for transiently transfecting cells in culture may comprise using chemical transfection agents. Nonlimiting examples of chemical transfection agents include, for example PEI, poly(propylene imine), DEAE-dextran, activated dendrimers, calcium phosphate, and cationic lipids. See, e.g. Kingston, R. E., et al., (2003). In some aspects, the transfection agent may include a commercially available transfection agent, such as PEIpro (POLYPLUS), PEIpro-HQ (POLYPLUS), PEIpro-GMP (POLYPLUS), PEI MAX (POLYSCIENCES), LIPOFECTAMINE2000 (INVITROGEN), TRANSFECTIN (BIO RAD), FectoVIR® (POLYPLUS) and similar.

In some aspects, the transfection agent comprises an inorganic transfection agent such as calcium phosphate. Though calcium phosphate (CaP) is particularly attractive due to its low cost and low cytotoxicity, CaP-mediated transfection is known to be hardly reproducible. A possible root cause of the variability is the rapid nucleic acid/CaP particle growth, which may be overcome using the continuous flow method described herein.

When CaP is used in methods as described herein, the nucleic acids may be blended with either the calcium source (such as a calcium salt) or the phosphate source. For example, where the plasmid(s) are blended with the phosphate source in one reservoir that is connected to the first inlet of the mixing fluidic device, and the calcium source is placed in a second reservoir connected to the second inlet of the mixing fluidic device. Upon mixing, the calcium phosphate is formed, which binds the plasmids and forms nucleic acid non-viral vector particles. Information on calcium phosphate-mediated transfection can be found in SPIZIZEN, J., REILLY, B. E., and EVANS, A. H. (1966). Microbial transformation and transfection. Annu. Rev. Microbid. 20, 371). As another example, the plasmid(s) may be blended first with the calcium source for some protocols, and then contacted with the phosphate source.

In some aspects, the transfection agent is PEI. PEI is understood to be a variable molecule, available in many forms and molecular weights, all of which are understood to be effective for the formation of a transfection complex and the successful cellular delivery thereof. In some aspects, the PEI has a molecular weight (MW) of between about 10 and 500 kDa.

In some aspects, the transfection agent is present in the TAS at a concentration that is with respect or controlled by the concentration of nucleic acid in the NAS. For example, as set forth in the working examples, ~2.6 µg of DNA/mL was prepared in the NAS. The TAS with a 1:2 desired weight:weight ratio would therefore require ~ 5.2 µg transfection agent/mL.

In some aspects, the transfection agent is provided in the TAS. The TAS may include a cell medium as described herein and are generally well understood in the art. The cell medium may additionally be supplemented, such as with antibiotics, cytokines, growth factors, hormones, saccharides, or similar. In some aspects, the cell medium to dilute the TAS contains no additional supplements.

### Cell Reactor or Bioreactor and Medium

Any suitable cell reactor or bioreactor may be used with methods according to embodiments described herein. In some aspects, the bioreactor is a perfusion bioreactor. In some aspects, the perfusion cell reactor is configured to have a flow of media through the reaction space, while also being configured to prevent cell escape, such as through a mesh or similar. In some aspects, the cells are adherent and the flow of the media will not largely cause cells to attempt to join the recirculating flow of media. A nonlimiting example of a perfusion bioreactor is the Corning® Ascent™ bioreactor (Corning Incorporated, Corning, NY). In some aspects, the bioreactor is a fixed-bed bioreactor. A nonlimiting example of a fixed-bed bioreactor is the iCELLis bioreactor (iCELLis). In some aspects, the bioreactor comprises packed-plain fibers, hollow fibers, packed or rolled fiber mesh, or packed-beds. In some aspects, the bioreactor comprises a wave reactor.

In aspects, a cell culture vessel or bioreactor used with methods as described herein is configured for adherent cell culture.

In aspects, a cell culture vessel or bioreactor used with methods described herein is configured for a suspension cell culture. For example, if a suspension reactor is used with methods according to embodiments described herein, the reactor may be equipped with a cell strainer or any other device that maintain the cells within the vessel during perfusion.

In aspects, the cell culture reactor includes one or more eukaryotic cells for transfection. In some aspects, the cells are of a mammalian origin. In some aspects, by way of example, the cells are chosen from human embryonic kidney (HEK) cells, Chinese hamster ovary (CHO) cells, HaCaT cells, Jurkat cells, HeLa cells, HT1080 cells, COS cells, BHK cells, LnCaP cells, MCF7 cells, NIH3T3 cells, VERO cells, PerC.6 cells, NSO cells, HEPG2 cells, A549 cells, HepG2 cells, K562 cells, LnCaP cells, PC-12 cells, PC-3 cells, and MDCK cells. It will be appreciated that other mammalian cell types can also be utilized as well. Similarly, non-mammalian cells can be used. For example, insect cells, such as Sf9 cells, can also be transfected. It will be appreciated that the cell type is not a limiting factor as the ability for the formed nucleic acid-transfection agent complex is generally readily capable of introducing the nucleic acid into the interior of cells.

In aspects, the cell reactor is operably connected with a media stock reservoir to allow for a continuous perfusion of media to the cells within the cell reactor. The main considerations for the media stock reservoir is cleanliness, non-reactivity, and sufficient volume to absorb the added NAS and TAS to the medium therein during the transfection process. In some aspects, the medium is a cell culture media, as a Dulbecco’s minimal essential medium (DMEM), DMEM/F12 Medium, Iscove’s modified Dulbecco’s medium (IMDM), F17 medium, RPMI 1640 medium, Ham’s medium, Ames’ medium, Sf-9 medium, OptiMEM, FreeStyle™ CHO, CHO-S-SFM II, CD-Forti CHO™, Power-CHO™ 1, Pro CHO™4, Free Style™ 293, Balan CD™ HEK 293, Free Style™ F17, Expi 293™, HyClone™ HyCell™° TransFx™-H, or similar.

In aspects, the medium may be supplemented as is understood in the art, such as with fetal bovine serum (FBS), antibiotics such as penicillin and/or streptomycin, growth factors, cytokines, hormones, glutamine, or similar.

### EXAMPLES

Embodiments of the present disclosure are further described below with respect to certain exemplary and specific embodiments thereof, which are illustrative only and not intended to be limiting.

### Example 1

Example 1 illustrates the transfection of HEK 293T cell cultured in an Ascent™ (Corning Incorporated, Corning, NY) small scale perfusion bioreactor using polyplex prepared by means of a Low Flow AFR (Corning Incorporated, Corning, NY) passive mixer according to the methods described herein or prepared in batch.

In the experimental setup for the Low Flow AFR mixer, two syringe pumps were connected to two inlets of a Low Flow AFR mixer (Corning Incorporated, Corning, NY) having an internal volume of 0.48 ml, for which the outlet was connected to the inlet at the bottom of an Corning® Ascent™ perfusion reactor cartridge (Corning Incorporated, Corning, NY). One syringe contained the plasmids mix and the other one contained the PEI solution.

### Cell Culture and Handling

All experiments were performed using adherent HEK293T cells cultivated in IMDM medium (Gibco) supplemented with 10% FBS (Gibco), 1x GlutaMAX™-I (Gibco) and 1000U/mL Penicillin, 1000 µg/mL Streptomycin (Penicillin-Streptomycin, Gibco). Cells were maintained adherent on tissue culture treated surfaces at 37° C. and 5% CO₂. They were subcultured twice a week using 0.25% Trypsin with 0.1% EDTA (Gibco). Only cells from passages less than 10 were used.

Three days prior transfection, HEK293T cells were seeded at about 8300 cells/cm² in the reactor. Cells were added to the media stock reservoir and perfused through the mesh reactor. Cell seeding was monitored and complete in around 3h. A media exchange was performed one hour before transfection.

### DNA and PEI Solution Preparation

The helper-free system, consisting of three plasmid DNA (pAAV-MCS-GFP, pAAV-RC2 and pHelper) that allow the production rAAV virions by transient transfection, was purchased from Cell Biolab (#AAV-400). One of the plasmids carries a GFP sequence that allows to evaluate transfection efficacy. All plasmids were amplified in Escherichia coli and isolated using a Maxi prep plasmid QIAGEN kit.

Before transfection, the three plasmids were combined at a ratio 1:1:1 (mass) in unsupplemented IMDM in order to achieve 0.18 µg DNA/cm² of the mesh reactor and ~2,6 µg DNA/mL in the total volume. Typically, 144 µg of each plasmid were diluted in unsupplemented IMDM for a total volume of 6 mL. The solution was mixed briefly by vortex, kept at room temperature used within 15 min.

As transfection agent, PEIpro® (Polyplus®) was used at a ratio DNA:PEI of 1:2 (weight:weight) and N/P=15. PEIpro was mixed with unsupplemented IMDM. Typically, 864uL PEIpro was diluted in unsupplemented IMDM for a total volume of 6 mL. The solution was mixed briefly by vortex, kept at room temperature, and used within 15 min.

### Cell Transfection

For batch transfection, an equal volume of DNA solution and PEIpro solution were mixed briefly by vortex. Typically, 6 mL of PEIpro solution was added to 6 mL of DNA solution and mixed by seven pulses of vortex. The solution was then kept for 10 min at room temperature with no agitation to allow polyplex formation. After 10 min, the polyplex solution was added to the media stock reservoir of the reactor and allowed to perfuse the mesh reactor for cell transfection for 24 hours.

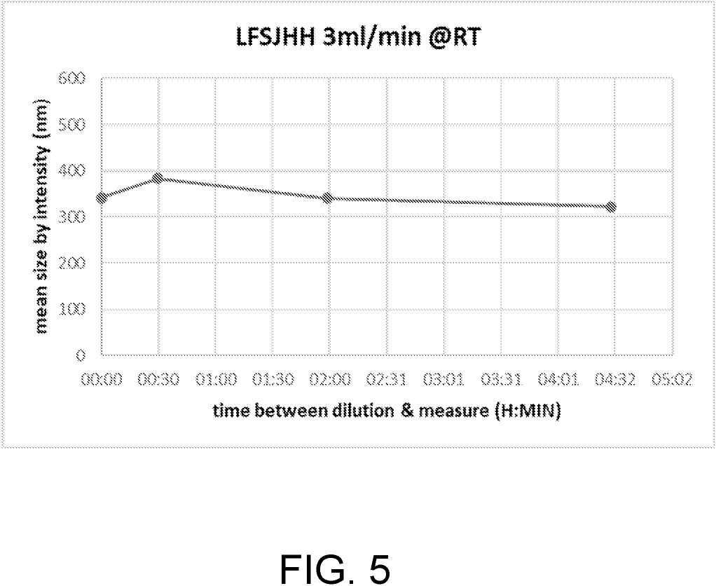

For AFR Low Flow transfection, sterile syringes were filled with the DNA solution and the PEIpro solution and connected to the AFR Low Flow reactor which itself is connected to the cell reactor (see scheme of the set up in FIG. 1). The mixing system was started and typically a total of 3 mL/min flow rate was used (1.5 mL/min for each syringe). An identical volume of polyplex solution was added to the mesh reactor compared to the batch transfection. The polyplex solution perfused the mesh reactor for 24h. FIG. 5 is showing the hydrodynamic diameter of the polyplex particles determine by DLS as function of time once diluted in the culture media. The graph shows clearly that the size of polyplex particles is highly stable. The average hydrodynamic diameter of the polyplex is 300-400 nm.

At 24h after transfection, a media exchange was performed for both batch and AFR Low Flow transfections.

### Polyplex Size Measurement

Polyplex size and surface charges was assessed using a Zetasizer Nano ZS. Polyplex solution from each transfection method were sampled either at the end of the 10 min incubation or right before entry in the mesh reactor system and diluted in complete IMDM, similarly to the dilution in the mesh reactor. For polyplex size and surface charges evolution assessment during time, polyplex were kept in similar conditions as in the mesh reactor (37° C.) and measured at the appropriate time points.

### GFP Fluorescence Observation by Microscopy

At 72h post-transfection, mesh reactors were opened, and some meshes at different area of the reactor were sampled for observation. The meshes were kept in complete IMDM in a petri dish and observation realized with an Olympus inverted fluorescent microscope. After observation, meshes were returned to the reactor for further analysis.

### GFP Fluorescence Quantification

At 72h post-transfection, the media was removed from the reactor and a PBS wash was performed. Cells were then dissociated from the mesh with a 45 min Accutase treatment and collected by centrifugation. Total cell amount for each sample was determined manually using a Malassez cell chamber. For each sample, GFP positive cells as well as mean fluorescence intensity were analyzed by flow cytometry using a BD Accuri C6 Plus system (BD Biosciences). Non-transfected cells were used as a negative control.

### AAV2 Capsid (cAAV2) Titration

Total cells from the reactor were pelleted. Lysis buffer (2 mM Tris-HCl, 150 mM NaCl, 0.5% sodium deoxycholate, 495U benzonase) was added to the pellet and cell were lysed at 37° C. for 30 min. Cell lysate were centrifuged at 21000 g for 5 min to pellet cell debris.

AAV2 capsids titration was then performed using the AAV2 titration ELISA (#PRATV, PROGEN) following manufacturer recommendation.

Sample absorbance was measured using a BioTeck Gen5 and AAV2 titer determined according to the standard curve.

### AAV2 Genome (gAAV2) Quantification

Total cells from the reactor were pelleted. Cell lysis and gAAV2 were quantified using the Takara AAVpro titration kit (#6233, Takara).

Briefly, cells were lysed using the lysis buffer provided in the kit. Cell lysate were treated with DNase followed by DNase inactivation and capsid lysis. AAV2 viral genomes were quantified by quantitative PCR using the agents and methods provided and recommended by the Takara kit. The quantitative PCR was performed using a QuantStudio 6 Pro (Applied Biosystems).

### Data Analysis

Data were analyzed using the GraphPad Prism software.

### Polyplex Size and Surface Charges

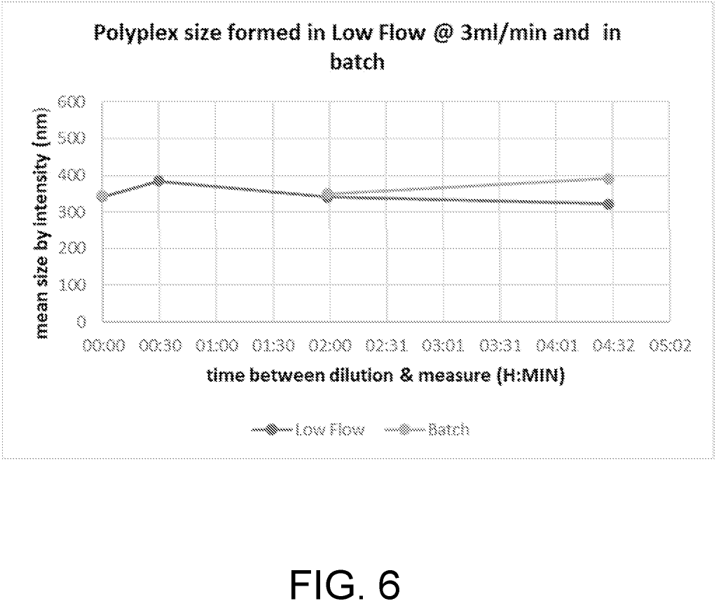

Polyplex size and surface charges was assessed using a Zetasizer Nano ZS. Polyplex solution from each transfection method were sampled either at the end of the 10 min incubation for batch or right before entry in the mesh reactor system for Low Flow preparation and diluted in complete IMDM, similar to the dilution in the mesh reactor. Polyplex size measurement and size evolution assessment during time (at 37° C.) are presented in FIG. 6 for batch and Low Flow preparation methods. Sizes are very close around 350 nm for both preparation methods and there is no significant evolution of size with time.

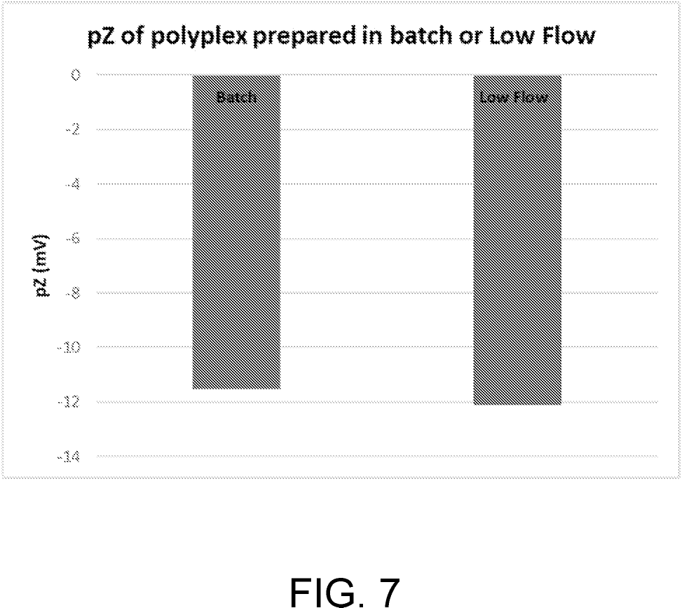

Potential zeta measurement of complexes formed with both preparation methods are presented in FIG. 7. There is no significant difference between two preparation methods with potential zeta being close to -12 mV.

### Comparison of Batch Transfection Method and AFR Low Flow Transfection Method

For both methods, identical PEIpro solution and DNA solution with the helper free system for AAV2 production were prepared with a DNA:PEI ration of 1:2.

The solutions were mixed briefly and allowed to stand for 10 min prior addition to the cell reactor for the Batch method. In the AFR Low Flow method, both solutions were connected to the AFR reactor in which they were mixed and then directly added to the cell reactor (direct connection).

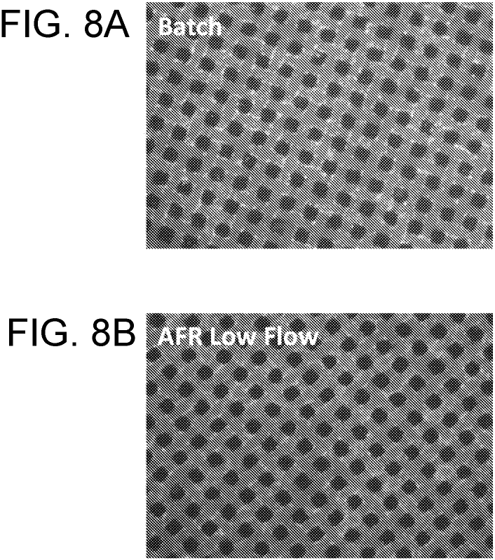

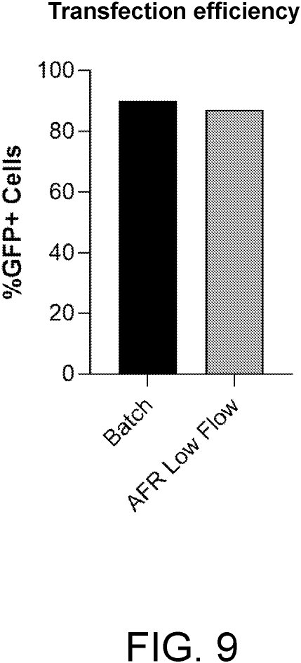

At 72h post-transfection, GFP expression was observed and analyzed as well as AAV2 titer determined. In both methods, cells are homogenously adherent on the mesh and expressing a similar lever of GFP (FIG. 8). A flow cytometry analysis indicated that in both methods, a similar quantity of GFP positive cells were present in each sample suggesting a similar level of transfection for both the Batch and the AFR Low Flow methods (FIG. 9).

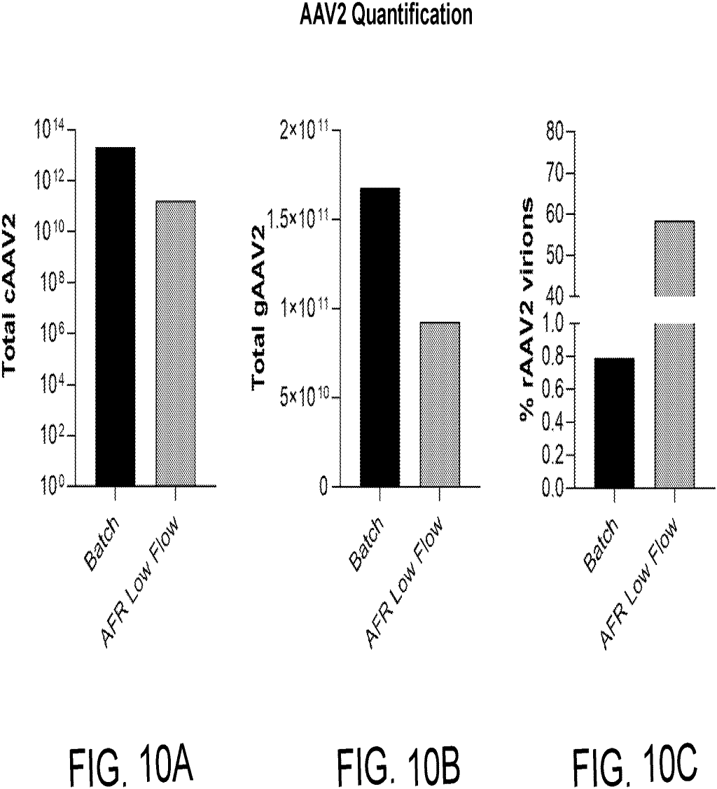

The cells were then lysed and treated to characterize the quality of the recombinant adeno-associated virus (rAAV2) produced. The quantity of total AAV2 capsid (cAAV2) produced was analyzed on one side and specifically the quantity of AAV2 viral genomes (gAAV2) on the other side. AAV2 capsids produced were about 20-fold higher in the Batch sample compared to the AFR Low Flow sample. However, AAV2 viral genomes produced were only 1.4-fold higher in the Batch sample compared to the AFR Low Flow method, indicating that both methods led to a similar amount of rAAV2 virion (full capsids) production (FIG. 10).

Moreover, the data indicate that the level of rAAV2 virion is about 8.57% with the AFR Low Flow method, while it is only about 0.8% with the Batch method. Such a strong increase of rAAV2 virions will then facilitate downstream rAAV2 purification methods.

Altogether, the data indicate that both the batch and the AFR Low Flow transfection method lead to a similar level of cell transfection and allow the production of AAV2 capsids and virions. However, although a similar level of transfection and rAAV2 virion are obtained, the level of rAAV2 virions is significantly higher with the AFR Low Flow method compared to the Batch method, facilitating downstream virion purification. In addition, the process described in the methods of the present disclosure made in flow can be readily scaled-up, whereas the batch process cannot. That clearly show the advantage of the method of the present disclosure in terms of industrialization.

### Example 2

Example 2 illustrates a comparison between T junction, AFR, and Batch methods, particularly the comparison of polyplex size and surface charges between the Batch method, AFR Low Flow method, and the T-junction method.

### Experimental Set-Up

The system set up, cell culture, and polyplex preparation were handled as described earlier with respect to Example 1 for Batch and AFR Low Flow conditions.

For the T-junction method, the AFR Low Flow reactor was replaced by a classical T-junction with no other parameter change. In this setup, the T-junction plays the role of passive mixer (T-mixer).

### Polyplex Size and Surface Charges

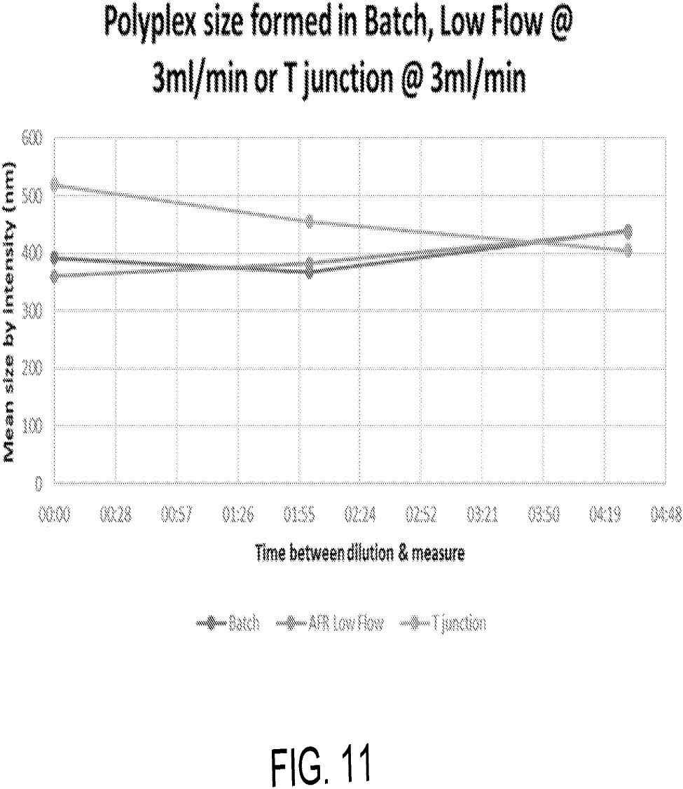

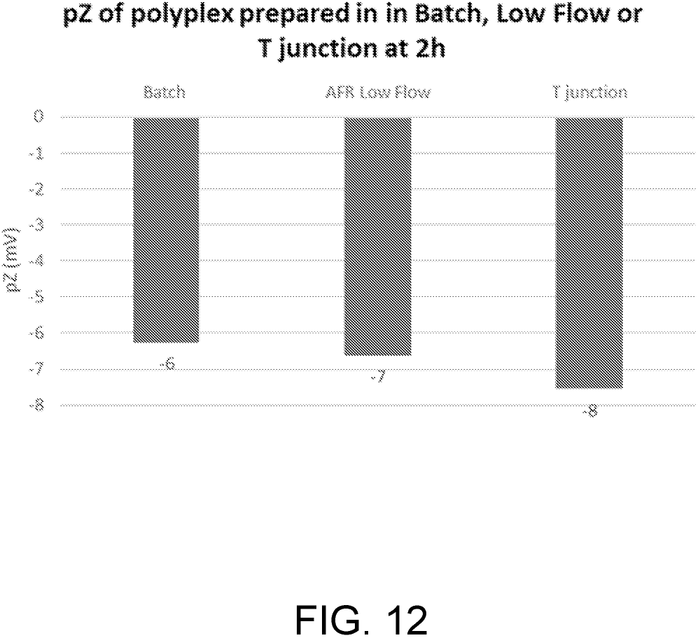

Polyplex size and surface charges were assessed using a Zetasizer Nano ZS and handled as described earlier. Although polyplex size was slightly higher for the T junction sample initially, its size decreased with time and stabilized at a similar level as the batch and AFR samples at about 3 hours. As previously determined, batch and AFR samples are of similar size and stable over time. Potential zeta measurement of polyplex generated in all three methods are similar. Polyplex size and potential zeta are presented in FIG. 11 and FIG. 12.

### Comparison of Batch Transfection Method, AFR Low Flow and a T Junction Transfection Method

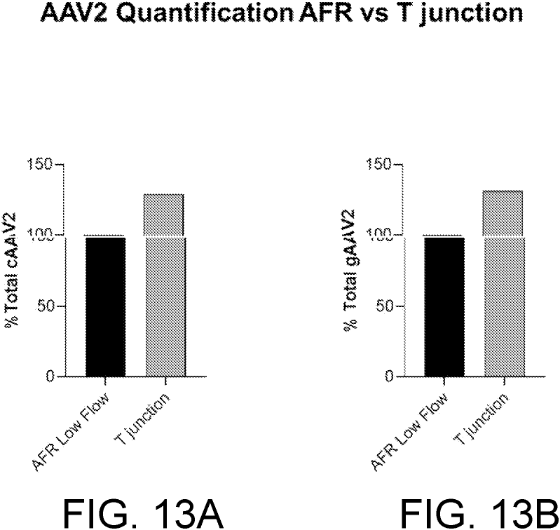

As described earlier, AAV2 production was assessed at 72 hours after transfection both for total viral genome (gAAV2) and total capsid (cAAV2) production. These data are presented in FIG. 13. Interestingly, the T junction method led to a slightly higher capsid production (cAAV2) and viral genomes (gAAV2) than the AFR method.

Altogether, these data indicate that in this set up and experimental conditions, polyplex mixing by an AFR reactor or T junction lead to a similar cAAV2 ang gAAV2 production.

### Example 3

Example 3 illustrates a comparison of AAV2 production variability between the Batch and the AFR method across ten independent experiments.

### Experimental Set-Up

The system set up, cell culture and polyplex preparation in the Batch method were handled as described earlier in Example 1.

For AFR Low Flow transfection, as in Example 1, sterile syringes containing the DNA solution on one hand and the PEIpro solution on the other hand, were connected to the AFR Low Flow reactor. This AFR Low Flow reactor was directly connected to the cell reactor by a tube with a specific length to allow for approximately two minutes residence at the mixing speed. The mixing system was initiated and for these series of experiments a total of 10 mL/min flow rate was used (5 mL/min for each syringe). Similar to example 1, an identical volume of polyplex solution was added to the mesh reactor compared to the batch transfection. Following the transfection, the polyplex solution perfused the mesh reactor for 24h in both the batch and the AFR Low Flow method.

### Comparison of AAV2 Production Variability Between the Batch and the AFR Method

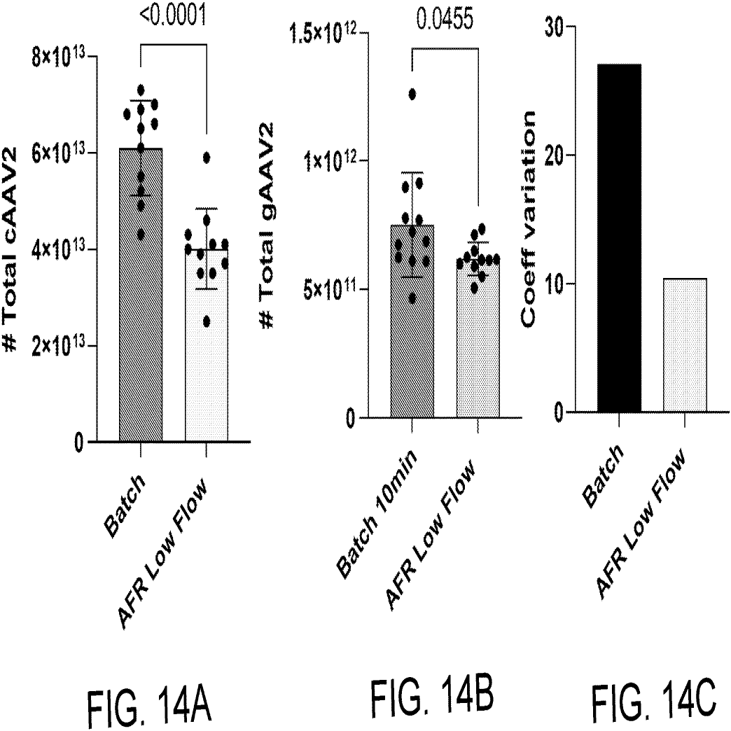

Here ten independent experiments were performed. Sample were processed as described earlier and data pooled to assess AAV2 production variability between the Batch and AFR method. As described in example 1, for each experiment, AAV2 production was assessed at 72 hours transfection for total viral genome (gAAV2) and total capsids (cAAV2) production. These data are presented in FIG. 14. There are slightly more total AAV2 capsid produced using the batch method with a mean cAAV2 produced of about 6.28×10¹³ while it is about 4.01×10¹³ using the AFR Low Flow method. Surprisingly, there is no strong difference in AAV2 viral genome produced between these two methods with about 7.51×10¹¹ gAAV2 produced using the batch method and 6.19×10¹¹ gAAV2 produced using the AFR Low Flow method and at the limit of statistical significance with a p value of 0.0455. More interestingly, the production of total AAV2 viral genome is more reproducible with the AFR Low Flow method with a coefficient of variation of about 10%, while it is around 27% with the batch method.

Altogether, these data indicate that even without a static phase of incubation time of at least ten minutes recommended in the literature for polyplex formation and maturation, the AFR Low Flow method allow to produce a similar level of AAV2 viral genome but with a higher level of reproducibility from one experiment to the next compared to the batch method.

### Example 4

Example 4 illustrates a comparison of AAV2 production according to the polyplex injection site into the bioreactor either at the bottom of the bioreactor or at the top of the bioreactor.

### Experimental Set-Up

The system set up, cell culture and polyplex preparation in the Batch method were handled as described earlier in Example 1.

For AFR Low Flow transfection, as in Example 1 and 3, sterile syringes containing the DNA solution on one hand and the PEIpro solution on the other hand, were connected to the AFR Low Flow reactor. As in Example 3, the AFR Low Flow reactor was directly connected to the bioreactor system by a tube with a specific length to allow for approximately two minutes residence at the mixing speed of 10 mL/min total flow rate. However, here the connection to the bioreactor was either through the bottom or the top of the top of the bioreactor. Through the connection from the bottom of the bioreactor, the polyplex solution perfused first the fixed bead reactor containing the cells before reaching the media containing vessel (FIG. 1). While through the connection from the top of the bioreactor, the polyplex solution goes first into the media containing vessel and is diluted in the media before reaching the cells in the bioreactor (FIG. 2A). Similarly, the polyplex solution prepared using the Batch method was either injected through the connection from the bottom of the bioreactor or through the connection from the top of the bioreactor.

As is Examples 1 and 3, an identical volume of polyplex solution was added to the mesh reactor compared to the batch transfection. Following the transfection, the polyplex solution perfused the mesh reactor for 24h in both the batch and the AFR Low Flow method.

### Comparison of AAV2 Production According to the Site of Polyplex Solution Injection

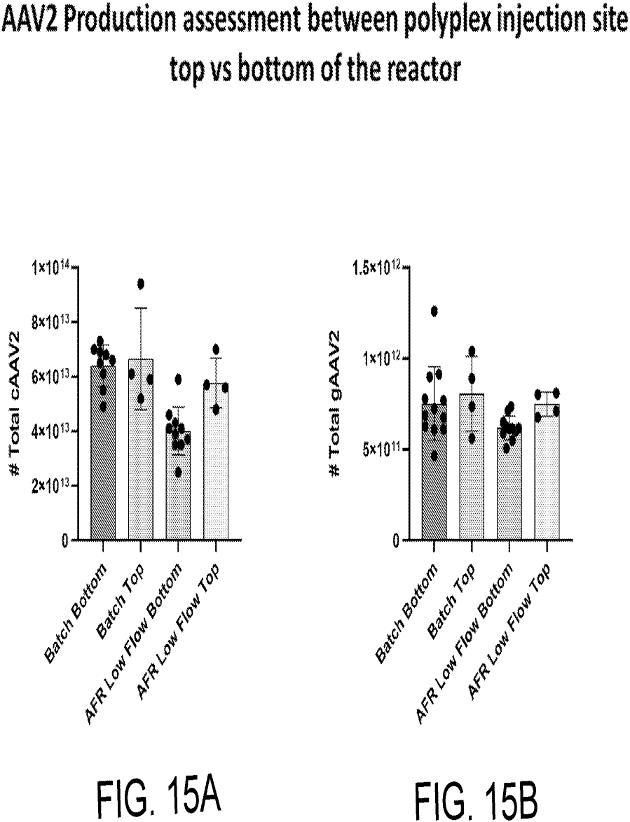

Here, a comparison of how the site of polyplex solution into the bioreactor can influence AAV2 production was carried out. At least four independent experiments were performed, and samples processed as described earlier. As described in Example 1 and 3, for each experiment, AAV2 production was assessed at 72 hours transfection for total viral genome (gAAV2) and total capsids (cAAV2) production. These data are presented in FIG. 15. Interestingly, for polyplex prepared using the Batch sample, a similar level of total AAV2 capsids (cAAV2) and total AAV2 viral genomes (gAAV2) were produced in both the bottom and top injection site into the bioreactor system. For polyplex prepared using the AFR Low Flow method, a slight increase of total AAV2 capsids (cAAV2) from 4.1x1013 cAAV2 to 5.77x1013 cAAV2 were produced when connected through the top injection site into the bioreactor. No change was observed in the total AAV2 viral genome (gAAV2) production. In addition, as shown in Example 3, similar levels of AAV2 viral genome were produced by polyplex prepared in both the Batch and the AFR Low Flow method.

Altogether, these data indicate that polyplex solution can enter the bioreactor system either by the bottom or the top with no consequence on the AAV2 viral genome production.

### Example 5

Example 5 illustrates a comparison of AAV2 production according to the tube length between the AFR Low Flow reactor and the bioreactor system. This tube length will determine a specific residence time of the polyplex.

### Experimental Set-Up

The system set up, cell culture and polyplex preparation in the Batch method were handled as described earlier in Example 1. As described previously, after mixing the DNA with the PEIpro, polyplex were incubated for 10 min at room temperature in static condition before injection into the bioreactor system. In addition to this sample, another batch sample was produced but with only one minute incubation time to assess the effect of polyplex incubation time on AAV2 production.

For AFR Low Flow transfection, as in Examples 1, 3, and 4, sterile syringes containing the DNA solution on one hand and the PEIpro solution on the other hand, were connected to the AFR Low Flow. As in Example 3 and 4, the AFR Low Flow reactor was directly connected to the bioreactor system by a tube but here with tubing length was varied (tubing diameter was constant and with laminar flow) between samples leading to various residence times from seven seconds to four minutes twenty seconds. For all AFR Low Flow samples, the mixing speed was of 10 mL/min total flow rate. In these set of experiments, the connection to the bioreactor was always through the top of the bioreactor. The polyplex solution prepared using the Batch method also injected through the connection from the top of the bioreactor.

As in Examples 1, 3, and 4, an identical volume of polyplex solution was added to the mesh reactor compared to the batch transfection. Following the transfection, the polyplex solution perfused the mesh reactor for 24h in both the batch and the AFR Low Flow method and AAV2 production was assessed 72 hours after transfection.

Comparison of AAV2 production according to the length of the tubing between the AFR Low Flow and the bioreactor system

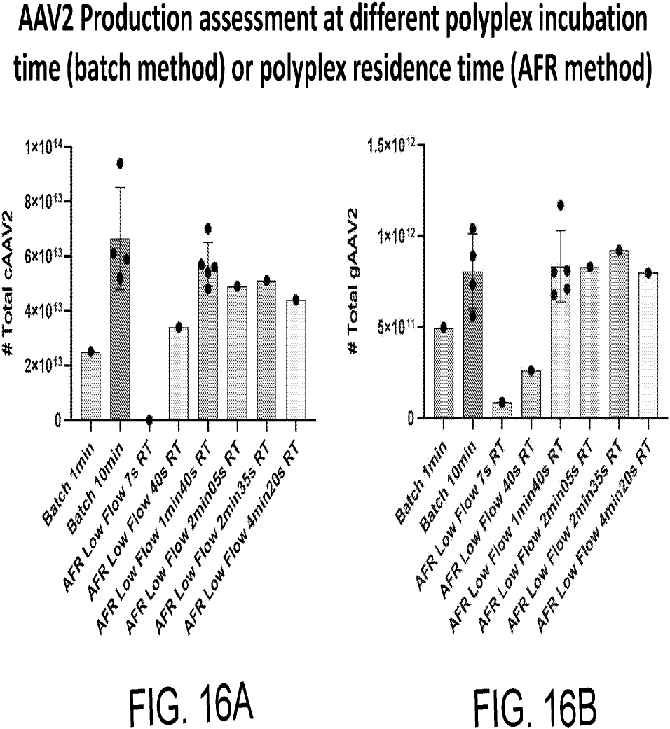

In this set of experiments, how the tube length and therefore the polyplex residence time in the AFR method as well as the polyplex incubation time in the batch method affect cell transfection was assessed, measured by AAV2 production. These data represent values obtained by five independent experiments. As described previously, for each experiment, AAV2 production was assessed for total viral genome (gAAV2) and total capsids (cAAV2) production. These data are presented in FIG. 16. As expected, in the batch method, polyplex incubation time is an important parameter as AAV2 capsids and viral genome produced were both lower with only one minute incubation time compared to the ten minutes incubation time. In the AFR method, a seven or forty second tube length led to a lower AAV2 capsid and viral genome production then longer tube length. Interestingly, a tube length leading to a one minute forty second residence time led to a similar level of AAV2 capsids and viral genome production than the batch method. And surprisingly, increasing further the tube length up to four minutes twenty second residence time did not further increase AAV2 production.

Altogether, these data indicate that both polyplex incubation time in the batch method and polyplex residence time in the AFR method have an impact on AAV2 production. More interestingly, in the AFR method, there seems to be an optimal tube length and polyplex residence time for AAV2 production of one minute forty seconds at the 10 mL/min mixing flow rate for the low flow small scale model.

It will be appreciated that the various disclosed embodiments may involve particular features, elements, or steps that are described in connection with that particular embodiment. It will also be appreciated that a particular feature, element, or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

All scientific and technical terms used herein have meanings commonly used in the art unless otherwise specified. The definitions provided herein are to facilitate understanding of certain terms used frequently herein and are not meant to limit the scope of the present disclosure.

As used herein, “have,” “having,” “include,” “including,” “comprise,” “comprising,” or the like are used in their open-ended sense, and generally mean “including, but not limited to.”

Ranges may be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been described in the Detailed Description, it should be understood that the disclosure is not limited to the disclosed embodiments, but is capable of numerous rearrangements, modifications and substitutions without departing from the disclosure as set forth and defined by the following claims.

What is claimed is:

1. A method for preparing transfected eukaryotic cells comprising:
operably connecting a nucleic acid solution (NAS) and a transfection agent solution (TAS) to a passive mixing module through two separate inlets, with a single outlet operably connected to a cell reactor, wherein the NAS comprises a nucleic acid at a first concentration and wherein the TAS comprises a transfection agent at a second concentration;
providing the NAS at a first flow rate;
providing the TAS at a second flow rate;
providing a combined stream of the NAS and TAS from the single outlet to the cell reactor through a length of tubing; and,
perfusing the cell reactor with cell medium from a media stock reservoir into an inlet of the cell reactor and out of an outlet of the cell reactor, wherein cells reside within the cell reactor between the inlet and the outlet.

2. The method of claim 1, wherein the outlet of the cell reactor provides the cell medium back to the media stock reservoir to recirculate the cell medium.

3. The method of claim 1, wherein the outlet of the cell reactor is operably linked to the media stock reservoir to returning the cell medium thereto.

4. The method of claim 1, wherein the length of tubing is configured to provide a residency time of between 1 and 30 minutes before the combined stream reaches the cell reactor.

5. The method of claim 1, wherein the passive mixing fluidic module comprises a plurality of heart shaped mixing chambers in a series.

6. The method of claim 1, wherein the first flow rate and the second flow rate are the same.

7. The method of claim 1, wherein the first concentration and second concentration are configured to provide a ratio of mass of nucleic acid to weight of transfection agent of between 4:1 to 1:4.

8. The method of claim 7, wherein the ratio is 1:2.

9. The method of claim 1, wherein the nucleic acid comprises a plasmid.

10. The method of claim 9, wherein the plasmid encodes an adeno-associated virus (AAV).

11. The method of claim 10, wherein the AAV is genetically modified to express a peptide of interest.

12. The method of claim 10, wherein the AAV is genetically modified to express a nucleic acid of interest.

13. The method of claim 1, wherein the transfection agent solution comprises polyethylenimine (PEI), poly(propylene imine), DEAE-Dextran, polyarginine, dendrimers, calcium phosphate, ionizable or cationic lipids, lipid-like lipidoids, or combinations thereof.

14. The method of claim 1, wherein the combined stream is continuously perfused through the cell reactor for a period of time of between 30 minutes and 24 hours.

15. A system for providing continuous transfection of cells, comprising:
a nucleic acid solution (NAS) containment and a transfection agent solution (TAS) containment;
a passive mixing fluidic module comprised of two separate inlets and a single outlet, wherein a first inlet is operably connected to the NAS containment, the second inlet is operably connected to the TAS containment;
a cell reactor comprised of an inlet and an outlet with one or more cells therebetween, wherein the outlet of the passive mixing fluidic module is operably connected to the inlet of the cell reactor; and,
a media stock reservoir comprised of an inlet, an outlet, and a cell medium, wherein the outlet of the media stock reservoir is operably connected to the inlet of the cell reactor and the inlet of the media stock reservoir is operably connected to the outlet of the cell reactor.

16. The system of claim 15, wherein the outlet of the passive mixing fluidic module is operably connected to the inlet of the cell reactor through a length of tubing.

17. The system of claim 15, wherein the length of tubing is configured to provide a residency time for a combined stream of the NAS and the TAS of from about 1 to about 30 minutes before reaching the cell reactor.

18. The system of claim 23, wherein the outlet of the passive mixing fluidic module is operably connected to the inlet of the cell reactor via the media stock reservoir.

19. The system of any of claim 23, further comprising a first pump configured to pump NAS solution from the NAS containment toward the outlet of the passive mixing fluidic module and a second pump configured to pump TAS solution from the TAS containment toward the outlet of the passive mixing fluidic module.

20. The system of claim 30, further comprising a third pump configured to flow the cell medium from the outlet of the media stock reservoir toward the inlet of the cell reactor along the length of tubing.
